SEQUENCE LISTING (10 sequences)

SEQ ID NO: 1 - protein, 11 aa, Artificial sequence
  Synthetic peptide
CRVRIDGAPAC

SEQ ID NO: 2 - protein, 11 aa, Artificial sequence
  Synthetic peptide
CRVRIDNMPMC

SEQ ID NO: 3 - protein, 11 aa, Artificial sequence
  Synthetic peptide
CRVRINGQPQC

SEQ ID NO: 4 - protein, 11 aa, Artificial sequence
  Synthetic peptide
CRVKIDGRPMC

SEQ ID NO: 5 - protein, 11 aa, Artificial sequence
  Synthetic peptide
CRLKIDGMPRC

SEQ ID NO: 6 - protein, 11 aa, Artificial sequence
  Synthetic peptide
CRIKIDGEGQC

SEQ ID NO: 7 - protein, 11 aa, Artificial sequence
  Synthetic peptide
CRVYIDGVSVC

SEQ ID NO: 8 - protein, 11 aa, Artificial sequence
  Synthetic peptide
CRVIIDGRRMC

SEQ ID NO: 9 - protein, 11 aa, Artificial sequence
  Synthetic peptide
CRYNIDGRPQC

SEQ ID NO: 10 - protein, 11 aa, Artificial sequence
  Synthetic peptide
CRIRIDQRPAC

(19) United States
(12) Patent Application Publication
(10) Pub. No.: US 2006/0135434 A1
(43) Pub. Date: Jun. 22, 2006

(54) New peptides

(76) Inventor: Mari Ann Kulseth, Oslo (NO)

Correspondence Address: GE HEALTHCARE, INC., IP DEPARTMENT, 101 CARNEGIE CENTER, PRINCETON, NJ, 08540-6231, US

(21) Appl. No.: 10/540,064

(22) PCT Filed: Dec. 29, 2003
(86) PCT No.: PCT/NO03/00443

§ 371 Date: Jun. 22, 2005

(30) Foreign Application Priority Data
Dec. 30, 2002 (NO) 2002685

(51) Int. Cl. A61K 38/10; A61K 38/08

(52) U.S. Cl. 514/15; 530/328

(57) ABSTRACT

The present invention relates to new peptides for targeting to vascular endothelial growth factor receptor 2, VEGFR-2. The invention further relates to their use in therapeutically effective treatment as well as for diagnostic imaging techniques.

### FIELD OF INVENTION

The present invention relates to new peptides and their use in therapeutically effective treatment as well as for diagnostic imaging techniques. More specifically the invention relates to the use of such peptides for targeting to vascular endothelial growth factor receptor 2 (VEGFR2/KDR(kinase insert domain-containing receptor)/flk-1 (fetal liver kinase)) expressed on angiogenic endothelial cells, haematopoietic stem cells, endothelial precursor cells in the bone marrow, and several malignant cells. Contrast agents based on these peptides may thus be used for diagnostic imaging of for example malignant diseases, heart diseases, endometriosis, inflammation-related diseases and rheumatoid arthritis. Moreover such agents may be used in therapeutic treatment of these diseases through inhibition of angiogenesis. Further these peptides can be used in drug delivery by carrying a therapeutic agent to a diseased site or tissue.

### BACKGROUND OF INVENTION

New blood vessels can be formed by two different mechanisms: angiogenesis or vasculogenesis. Angiogenesis is the formation of new blood vessels by sprouting/branching from existing vessels. The primary stimulus for this process may be inadequate supply of nutrients and oxygen (hypoxia) to cells in a tissue. The cells may respond by secreting angiogenic factors, of which there is many; one example, which is frequently referred to, is vascular endothelial growth factor (VEGF). These factors initiate the secretion of proteolytic enzymes that break down the proteins of the basement membrane, as well as inhibitors that limit the action of these potentially harmful enzymes. The other prominent effect of angiogenic factors is to cause endothelial cells to migrate and divide. Endothelial cells that are attached to the basement membrane, which forms a continuous sheet around blood vessels on the contralumenal side, do not undergo mitosis. The combined effect of loss of attachment and signals from the receptors for angiogenic factors is to cause the endothelial cells to move, multiply, and rearrange themselves, and finally to synthesise a basement membrane around the new vessels. Vasculogenesis is the generation of new vessels by recruiting endothelial precursor cells from the bone marrow. Newly published data shows that vasculogenesis not only is restricted to fetal blood vessel formation, but also occurs in the adult as response to various conditions. The bone marrow derived endothelial precursor cells recruited are also expressing VEGFR2.

Angiogenesis is prominent in the growth and remodelling of tissues, including wound healing and inflammatory processes. Tumours must initiate angiogenesis when they reach millimetre size in order to keep up their rate of growth. Angiogenesis is accompanied by characteristic changes in the endothelial cells and their environment. The surface of these cells is remodelled in preparation for migration, and cryptic structures are exposed where the basement membrane is degraded, in addition to the variety of proteins which are involved in effecting and controlling proteolysis. In the case of tumours, the resulting network of blood vessels is usually disorganised, with the formation of sharp kinks and also arteriovenous shunts.

Inhibition of angiogenesis is considered to be a promising strategy for anti-tumour therapy. The transformations accompanying angiogenesis are also very promising as targets for diagnosis. An obvious example is malignant disease, but the concept also shows great promise in inflammation and a variety of inflammation-related diseases, including atherosclerosis. The macrophages of early atherosclerotic lesions are potential sources of angiogenic factors. These factors are also involved in re-vascularisation of infarcts in the myocardium.

Further examples of undesired conditions that are associated with neovascularization or angiogenesis implying the development or proliferation of new blood vessels, are shown below. Reference is also made in this regard to WO 98/47541.

Diseases and indications associated with angiogenesis are e.g. different forms of cancer and metastasis, e.g. breast, skin, colorectal, pancreatic, prostate, lung or ovarian cancer.

Other diseases and indications are inflammation (e.g. chronic), atherosclerosis, rheumatoid arthritis and gingivitis.

Further diseases and indications associated with angiogenesis are arteriovenous malformations, astrocytomas, choriocarcinomas, endometriosis, glioblastomas, gliomas, hemangiomas (childhood, capillary), hepatomas, hyperplastic endometrium, ischemic myocardium, Kaposi sarcoma, macular degeneration, melanoma, neuroblastomas, occluding peripheral artery disease, osteoarthritis, psoriasis, retinopathy (diabetic, proliferative), scleroderma, seminomas and ulcerative colitis. The malignant cells and the stroma cells upregulate proteins that are involved in the process of angiogenesis. Markers with different specificity are expressed on the endothelial cells. These markers include growth factor receptors such as VEGFR2. Immunohistochemical studies in combination with electron microscopy have demonstrated that VEGFR2 is expressed on the abluminal and luminal plasma membranes of vascular endothelial cells (Dvorak & Feng, 2001 J Histochem Cytochem, 49:419). VEGF produced by hypoxic tumour cells or stromal cells binds to the VEGFR2 on endothelial cells and stimulate angiogenesis. As complexes of VEGF and VEGFR2 are found predominantly on the abluminal side of the vascular endothelium, VEGFR2 available for targeting by circulating ligands is available at the luminal surface.

### THE PRESENT INVENTION

It has now been found a new peptide targeting the vascular endothelial growth factor receptor 2, VEGFR 2. This new peptide can be used as a therapeutic agent in a pharmaceutical formulation by inhibiting the angiogenesis in the diseased area/tissue.

Further the peptide can be coupled to a known therapeutic agent that will be carried to the diseased area/tissue by the targeting abilities of the new peptide.

One or more peptide can further be coupled to a chelating agent or a reporter moiety either by direct bonding or via a linker moiety to act as a diagnostic imaging agent or a therapeutic active agent.

### DETAILED DESCRIPTION OF THE INVENTION

The invention is described in the patent claims. Specific features of the invention are outlined in the following detailed description and the Examples.

The three letter abbreviations used for the amino acids have the following meaning:

Arg—Arginine

Asp—Aspartic acid

Cys—Cysteine

Hcy—Homocysteine

Gly—Glycine

Val—Valine

Tyr—Tyrosine

Hle—Isoleucine

Pro—Proline

Ser—Serine

Leu—Leucine

Lys—Lysine

Asn—Aspargine

Gln—Glutamine

Ala—Alanine

Met—Methionine

Glu—Glutamic acid

In a first aspect, the present invention provides a new peptide that targets VEGFR 2.

The new peptide comprising the amino acid sequence of formula (I)
Z¹-Arg-X²-X³-Ile-X⁵-X⁶-X⁷-X⁸-X⁹-Z²-Y¹ (Formula I)
wherein
X² is an amino acid selected from the group Val, Leu, Ile and Tyr
X³ is an amino acid selected from the group Arg, Lys, Tyr, Ile and Asn
X⁵ is an amino acid selected from the group Asp and Asn
X³ is an amino acid selected from the group Gly, Asn and Gin
X⁷ is an amino acid selected from the group Ala, Met, Gln, Arg, Glu and Val,
X⁸ is an amino acid selected from the group Pro, Gly, Ser and Arg
X⁹ is an amino acid selected from the group Ala, Met, Gin, Arg, Gly and Val
Z¹ represent an amino acid residue capable of forming a disulphide bond, preferably a cysteine or a homocysteine residue, or a residue capable of forming a thioether preferably the residue is Q-C(═O) wherein Q represents —(CH₂)n or —(CH₂)ₙ—C₆H₄ where n represents a positive integer 1 to 10 or is absent and
Z² represent an amino acid residue capable of forming a disulphide bond, preferably a cysteine or a homocysteine residue or is absent
Y¹ represents 1-10 amino acids or is absent
or pharmaceutically acceptable salts thereof.

More specific the new peptide comprises the amino acid sequence of formula (II)
Z¹-Arg-Val(Arg/Lys)Ile-Asp-Gly-X⁷-Pro-X⁹-Z²-Y¹ Formula (II)
wherein
X⁷ is an amino acid selected from the group Pro, Gly, Ser and Arg
X is an amino acid selected from the group Ala, Met, Gln, Arg, Gly and Val
Z¹ represent an amino acid residue capable of forming a disulphide bond, preferably a cysteine or a homocysteine residue, or a residue capable of forming a thioether preferably the residue is Q-C(═O) wherein Q represents —(CH₂)n or —(CH₂), —C₆H₄
where n represents a positive integer 1 to 10 or is absent and
Z² represent an amino acid residue capable of forming a disulphide bond, preferably a cysteine or a homocysteine residue or is absent
Y¹ represents 1-10 amino acids or is absent
or pharmaceutically acceptable salts thereof.

Further preferred are peptides comprising an amino acid sequence as follow:

| | |
|---|---|
| Cys-Arg-Val-Arg-Ile-Asp-Gly-Ala-Pro- | (SEQ ID NO 1) |
| Ala-Cys,, | |
| Cys-Arg-Val-Arg-Ile-Asp-Asn-Met-Pro- | (SEQ ID NO 2) |
| Met-Cys,, | |
| Cys-Arg-Val-Arg-Ile-Asn-Gly-Gln-Pro- | (SEQ ID NO 3) |
| Gln-Cys,, | |
| Cys-Arg-Val-Lys-Ile-Asp-Gly-Arg-Pro- | (SEQ ID NO 4) |
| Met-Cys,, | |
| Cys-Arg-Leu-Lys-Ile-Asp-Gly-Met-Pro- | (SEQ ID NO 5) |
| Arg-Cys,, | |
| Cys-Arg-Ile-Lys-Ile-Asp-Gly-Glu-Gly- | (SEQ ID NO 6) |
| Gln-Cys,, | |
| Cys-Arg-Val-Tyr-Ile-Asp-Gly-Val-Ser- | (SEQ ID NO 7) |
| Val-Cys,, | |
| Cys-Arg-Val-Ile-Ile-Asp-Gly-Arg-Arg- | (SEQ ID NO 8) |
| Met-Cys,, | |
| Cys-Arg-Tyr-Asn-Ile-Asp-Gly-Arg-Pro- | (SEQ ID NO 9) |
| Gln-Cys, | |
| or | |
| Cys-Arg-Ile-Arg-Ile-Asp-Gln-Arg-Pro- | (SEQ ID NO 10) |
| Ala-Cys,. | |

Viewed from another aspect the invention provides new peptide-based compounds as defined by formula (III).
V-L-Z Formula (III)
wherein the vector V is a peptide as defined above, L represents a bond, a spacer or linker and Z represents an antineoplastic agent, a reporter moiety or a group that optionally can carry an imaging moiety M.

The role of the linker L is to couple vector to reporter, and in the case where L is a spacer moiety the role of L is to distance the relatively bulky chelating agent from the active site of the peptide component. The spacer moiety L is also applicable to distance a bulky antineoplastic agent from the active site of the peptide.

A linker moiety may serve to link one vector to one reporter; alternatively it may link together more than one vector and/or more than one reporter. Likewise a reporter or a vector may be linked to more than one linker. Use in this way of a plurality of reporters (e.g. several linker-reporter moietes attached to one vector or several reporters attached to one linker itself attached to one vector) may enable the detectability of the contrast agent to be increased (e.g. by increasing its radioopacity, echogenicity or relaxivity) or may enable it to be detected in more than one imaging modality. Use in this way of a plurality of vectors may e.g. increase the targeting efficiency of a contrast agent or may make the contrast agent/therapeutic agent able to target more than one site, e.g. different receptors for an agent which has receptor heterogeneity.

The linker moiety L may be a simple bond or may be represented by other linkers well known in the art, e.g. as described in WO 01/77145 pages 23-27, the content of which are incorporated herein by reference.

Z can be represented by stabilised gas-filled microbubbles. In this aspect of the invention the compounds of formula (III) can be used for targeted ultrasound imaging. Each of the microbubbles may carry several vectors V.

Z can further be represented by a chelating agent of Formula (IV) 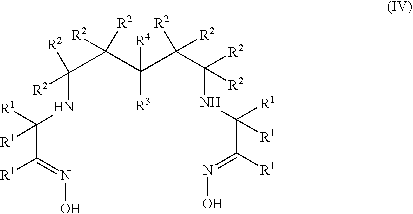
where:
each R¹, R², R³ and R⁴ is independently an R group;
each R group is independently H or C₁₋₁₀ alkyl, C₃₋₁₀ alkylaryl, C₂₋₁₀ alkoxyalkyl, C₁₋₁₀ hydroxyalkyl, C₁₋₁₀ alkylamine, C₁₋₁₀ fluoroalkyl, or 2 or more R groups, together with the atoms to which they are attached form a carbocyclic, heterocyclic, saturated or unsaturated ring, or can represent a chelating agent given by formulas a, b, c and d.

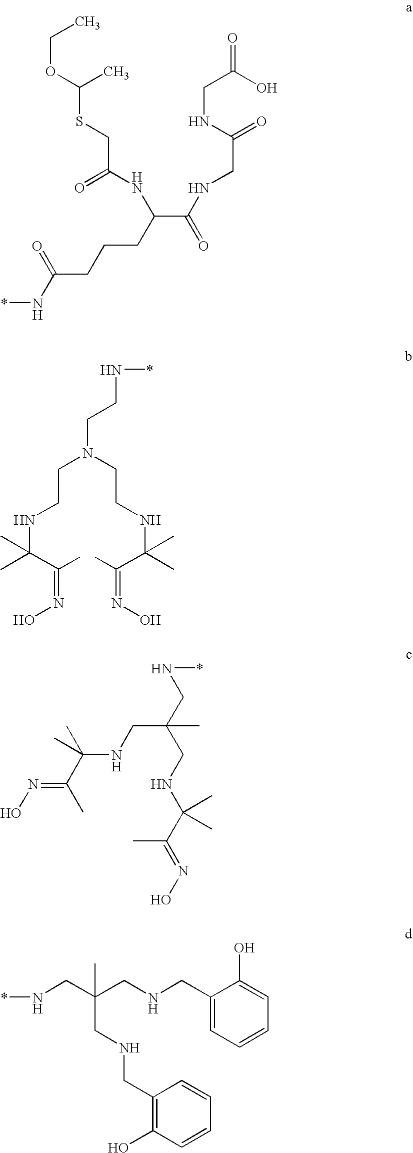

A preferred example of a chelating agent is represented by formula e.

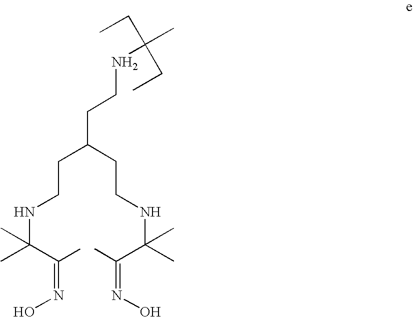

Conjugates comprising chelating agents of formula (IV) can be radio-labelled to give good radiochemical purity, RCP, at room temperature, under aqueous conditions at near neutral pH. The risk of opening the disulphide bridge of the peptide component at room temperature is less than at an elevated temperature. A further advantage of radio-labelling the conjugates at room temperature is a simplified procedure in a hospital pharmacy.

However the compounds defined in formula (III) may also comprise chelating agents, Z, as defined in WO 01/77145, Table I, pages 11-15.

In some aspects of the invention, Z comprises a reporter moiety M where said reporter moiety comprises a radionuclide. Further definitions of chelating agents are listed in WO 01/77145, Table I, pages 11-15, the content of which are incorporated herein by reference.

In one aspect of the present invention of formula (III) Z is represented by an antineoplastic agent. In this aspect the compound will target an angiogenic site associated with cancer and bring the antineoplastic agent to the diseased area. The antineoplastic agent may be represented by cyclophosphamide, chloroambucil, busulphan, methotrexate, cytarabine, fluorouracil, vinblastine, paclitaxel, doxorubicin, daunorubicin, etoposide, teniposide, cisplatin, amsacrine, docetaxel, but a wide range of other antineoplastic agents may also be used.

The peptide component of compounds of formula (III) may be in a cyclic configuration, i.e. by a disulphide bond or it may be linear.

The peptide component of the conjugates described herein has preferably no free amino- or carboxy-termini. This introduces into these compounds a significant increase in resistance against enzymatic degradation and as a result they have an increased in vivo stability as compared to many known free peptides.

The reporter moieties (M) in the contrast agents of the invention may be any moiety capable of detection either directly or indirectly in an in vivo diagnostic imaging procedure.

For MR imaging the reporter will either be a non zero nuclear spin isotope (such as ¹⁹F) or a material having unpaired electron spins and hence paramagnetic, superparamagnetic, ferrimagnetic or ferromagnetic properties; for light imaging the reporter will be a light scatterer (e.g. a coloured or uncoloured particle), a light absorber or a light emitter; for magnetometric imaging the reporter will have detectable magnetic properties; for electrical impedance imaging the reporter will affect electrical impedance; and for scintigraphy, SPECT, PET, and the like, the reporter will be a radionuclide.

Stated generally, the reporter may be (1) a chelatable metal or polyatomic metal-containing ion (i.e. TcO, etc), where the metal is a high atomic number metal (e.g. atomic number greater than 37), a paramagentic species (e.g. a transition metal or lanthanide), or a radioactive isotope, (2) a covalently bound non-metal species which is an unpaired electron site (e.g. an oxygen or carbon in a persistant free radical), a high atomic number non-metal, or a radioisotope, (3) a polyatomic duster or crystal containing high atomic number atoms, displaying cooperative magnetic behaviour (e.g. superparamagnetism, ferrimagnetism or ferromagnetism) or containing radionuclides.

Examples of particular preferred reporter groups (M) are described in more detail below.

Chelated metal reporters are preferably chosen from the group below; ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰³Pb and ¹⁴¹Ce.

The metal ions are desirably chelated by chelating agents on the linker moiety. Further examples of suitable chelating agents are disclosed in U.S. Pat. No. 4,647,447, WO89/00557, U.S. Pat. No. 5,367,080, U.S. Pat. No. 5,364,613, the content of which are incorporated herein by reference.

Methods for metallating any chelating agents present are within the level of skill in the art. Metals can be incorporated into a chelant moiety by any one of three general methods: direct incorporation, template synthesis and/or transmetallation. Direct incorporation is preferred.

Thus it is desirable that the metal ion be easily complexed to the chelating agent, for example, by merely exposing or mixing an aqueous solution of the chelating agent-containing moiety with a metal salt in an aqueous solution preferably having a pH in the range of about 4 to about 11. The salt can be any salt, but preferably the salt is a water soluble salt of the metal such as a halogen salt, and more preferably such salts are selected so as not to interfere with the binding of the metal ion with the chelating agent. The chelating agent-containing moiety is preferably in aqueous solution at a pH of between about 5 and about 9, more preferably between pH about 6 to about 8. The chelating agent-containing moiety can be mixed with buffer salts such as citrate, carbonate, acetate, phosphate and borate to produce the optimum pH. Preferably, the buffer salts are selected so as not to interfere with the subsequent binding of the metal ion to the chelating agent.

The following isotopes or isotope pairs can be used for both imaging and therapy without having to change the radio-labelling methodology or chelator ⁴⁷Sc₂₁; ¹⁴¹Ce₅₈; ¹⁸⁸Re₇₅; ¹⁷⁷Lu₇₁; ¹⁹⁹Au₇₉; ⁴⁷Sc₂₁; ¹³¹I₅₃; ⁶⁷Cu₂₉; ¹³¹I₅₃ and ¹²³I₅₃; ¹⁸⁸Re₇₅ and ^{99m}Tc₄₃; ⁹⁰Y₃₉ and ⁸⁷Y₃₉; ⁴⁷Sc₂₁ and ⁴⁴Sc₂₁; ⁹⁰Y₃₉ and ¹²³I₅₃; ¹⁴⁶Sm₆₂ and ¹⁵³Sm₆₂; and ⁹⁰Y₃₉ and ¹¹¹In₄₉.

Preferred non-metal atomic reporters include radioisotopes such as ¹²³I, ¹³¹I and ¹⁸F as well as non zero nuclear spin atoms such as ¹⁹F, and heavy atoms such as I.

In a further embodiment of this invention, the use of radioisotopes of iodine or fluorine is specifically contemplated. For example, if the peptide or linker is comprised of substituents that can be chemically substituted by iodine or fluorine in a covalent bond forming reaction, such as, for example, substituents containing hydroxyphenyl or p-nitrobenzoyl functionality, such substituents can be labelled by methods well known in the art with a radioisotope of iodine or fluorine respectively. These species can be used in therapeutic and diagnostic imaging applications. While, at the same time, a metal attached to a chelating agent on the same peptide-linker can also be used in either therapeutic or diagnostic imaging applications.

The compounds of formula (III) may be therapeutically effective in the treatment of disease states as well as detectable in in vivo imaging. Thus for example the vector on the reporter moieties may have therapeutic efficacy, e.g. by virtue of the radiotherapeutic effect of a radionuclide reporter of the vector moiety.

The present invention also provides a pharmaceutical composition comprising an effective amount (e.g. an amount effective for enhancing image contrast in in vivo imaging) of a compound of general formula (III) or a salt thereof, together with one or more pharmaceutically acceptable adjuvants, excipients or diluents.

The invention further provides a pharmaceutical composition for treatment of a disease comprising an effective amount of a compound of general formula (III), or a salt thereof, together with one or more pharmaceutically acceptable adjuvants, excipients or diluents.

Use of the new peptide and the compounds of formula (III) in the manufacture of therapeutic compositions (medicament) and in methods of therapeutic or prophylactic treatment, preferably treatment of cancer, of the human or animal body are thus considered to represent further aspects of the invention.

Viewed from a further aspect the invention provides the use of a compound of formula (III) for the manufacture of a contrast medium for use in a method of diagnosis involving administration of said contrast medium to a human or animal body and generation of an image of at least part of said body.

Viewed from a still further aspect the invention provides a method of generating enhanced images of a human or animal body previously administered with a contrast agent composition comprising a compound as defined by formula (III), which method comprises generating an image of at least part of said body.

Viewed from a further aspect the invention provides a method of monitoring the effect of treatment of a human or animal body with a drug to combat a condition associated with cancer, preferably angiogenesis, e.g. a cytotoxic agent, said method involving administering to said body an agent of formula (III) and detecting the uptake of said agent by cell receptors, preferably endothelial cell receptors and in particular VEGF receptors, said administration and detection optionally but preferably being effected repeatedly, e.g. before, during and after treatment with said drug.

These new compounds may be used in therapeutically effective treatments as well as for imaging purposes. Further these new compounds may be used for drug delivery purposes.

General Procedures

The abbreviations used have the following meanings:

Fmoc—9-fluorenylmethoxycarbonyl

Acm—acetamidomethyl

TFA—trifluoroacetic acid

ACN—acetonitril

RP-HPLC—reversed phase high pressure liquid chromatography

DMF dimethylformamide

NMM—N-methylmorpholine

DCM—dichloromethane

NMP—N-methylpyrrolidone

HBTU—2-(1H-benzotriazol-1-yl)-1,1,3,3-tetramethyluronium-hexafluorophosphate

HOBt—1-hydroxy-benzotriazole

DIEA—diisopropylethylamine

DTT—dithiotreitol

The peptides of the present invention can be synthesised using all the known methods of chemical synthesis but particularly useful is the solid-phase methodology of Merrifield employing an automated peptide synthesiser (J. Am. Chem. Soc., 85: 2149 (1964)). Typically, the desired sequences are assembled by solid-phase peptide synthesis. Standard procedures for the synthesis strategy employed for the examples of this invention are described in E. Atherton & R. C. Sheppard, “Solid phase peptide synthesis: a practical approach, 1989, IRL Press, Oxford.

For example, a synthesis resin with an acid-labile linker group, to which the desired protected C-terminal amino acid residue is attached by amide bond formation, is used. In the following examples 1 to 3, a so-called Rink amide AM resin with a (dimethoxyphenyl-aminomethyl)-phenoxy-derived linker was applied (Rink, H. Tetrahedron Lett. (1987), 30, 3787). Moreover an even more acid labile resin can be used, a so-called XAL-MBHA resin with a xanthenyl-derived linker (Han, Y.; Bontems, S. L.; Hegyes, P.; Munson, M. C.; Minor, C. A.; Kates, S. A.; Albericio, F.; Barany, G., J. Org. Chem. (1996), 61, 6326-6339).

The N^{α}-amino-protecting group is then removed and the second amino acid in the sequence is coupled using suitable condensation reagents. N^{α}-amino-deprotection and coupling cycles are then repeated in alternating steps until the sequence of interest is assembled. Amino acids with a temporary N^{α}-amino protecting group and permanent protecting groups for the functional side chains are employed. Generally, all reactive groups present (for example amino, hydroxyl, thiol and carboxyl groups) will be protected during peptide synthesis.

A wide range of protecting groups for amino acids is known (see, e.g., Greene, T. W. & Wuts, P. G. M. (1991) Protective groups in organic synthesis, John Wiley & Sons, New York). Thus, examples of amino protecting groups which may be employed include 9-fluorenylmethoxycarbonyl (Fmoc), benzyloxycarbonyl, t-butyloxycarbonyl, ect. When the peptide is built up from the C-terminal end, the N^{α}-amino-protecting group will need to be removed selectively prior to the next coupling step. One particularly useful group for such temporary amine protection is the Fmoc group which can be removed selectively by treatment with piperidine in an organic solvent. Carboxyl protecting groups which may be employed include for example t-butyl, benzyl, trityl, etc. The thiol protecting group used can be semi-permanent for example the p-methoxytrityl group or permanent including the trityl and acetamidomethyl groups. It will be appreciated that a wide range of other such groups are known in the art.

Finally the peptide is cleaved from the synthesis resin and the permanent side-chain protecting groups are removed, usually simultaneously as in examples 1 to 3 below. Alternatively, a synthesis resin is used which allows detachment of the peptide under mild conditions where the side-chain protecting groups are stable, affording protected peptides.

The invention is further illustrated by the following none limiting examples.

### EXAMPLES

### Example 1

cyclo-[CH₂CO-Arg-Val-Lys-Ile-Asp-Gly-Arg-Pro-Met-Cys]-Gly-Glu-Glu-Glu-Cys-NH₂

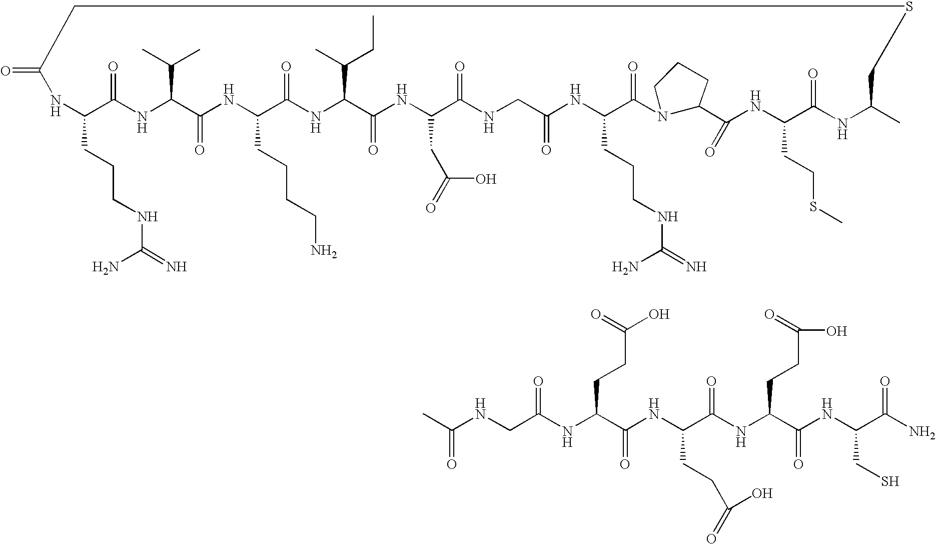

The peptidyl resin corresponding to the above sequence was assembled on a Rink Amide AM resin (0.64 mmol/g; from NovaBiochem) using an Applied Biosystems (perkin Elmer) model 433A peptide synthesizer. Fmoc deprotection was achieved with conductivity monitoring using 20% piperidine in N-methylpyrrolidone (NMP). The washing solvent was NMP. The residues (from the carboxyl terminus) were assembled on a 0.25 mmol scale using single couplings with a 4-fold molar excess of Fmoc-amino acids (1 mmol cartridges) and 2-(1H-benzotriazol-1-yl)-1,1,3,3-tetramethyluronium-hexafluorophosphate (HBTU)/1-hydroxy-benzotriazole (HOBt)/diisopropylethylamine (DIEA) in NMP using 2.5 hours coupling cycles. The amino acid-side chain protecting groups used were tert-butyl for Asp, Ser and Tyr, trityl for Cys¹⁰ and acetamidomethyl (Acm) for Cys¹⁵. The Gly residue coupled in position 6 was used with an additional N^{□}-protecting group (2-Fmoc-oxy-4-methoxybenzyl) in order to suppress aspartimide formation (Packman, L. C. 1995, Tetrahedron Lett. 36, p. 7523-7526).

Additional side-chain protecting groups used were 2,2,5,7,8-pentamethylchroman-6-sulphonyl for Arg, tert-butyloxycarbonyl for Lys and tert-butyl for Glu. The assembled peptidyl resin was then transferred to a manual nitrogen bubbler apparatus (Wellings, D. A., Atherton, E. (1997) in Methods in Enzymology (Fields, G. ed), 289, p. 53-54, Academic Press, New York). The N-terminus was Fmoc-deprotected and then bromoacetylated using a 10-fold molar excess of bromoacetyl bromide and N-methylmorpholine (NMM) in dimethylformamide (DMF) for 1 hour. The completed peptidyl resin was washed with DMF and dichloromethane (DCM) and dried in vacuo.

Simultaneous cleavage of the peptide from the resin and removal of the side-chain protecting groups (except the acetamidomethyl group) from the peptide, was effected by treating an aliquot of the peptidyl resin (0.125 mmol) with trifluoroacetic acid (TFA) containing 2.5% ethanedithiol, 2.5% water and 1% triisopropylsilane for 2 hours. The resin residue was filtered off and washed with small quantifies of neat TFA. The combined filtrate and washings were concentrated by rotary evaporation and then triturated with diethyl ether to obtain the crude peptide. The precipitate was isolated by centrifugation, washed with ether and then lyophilized from 50% acetonitrile (ACN)-0.1% aq TFA giving a crude product yield of 150 mg.

The linear crude peptide was cyclized by thioetherbridge formation, effected by stirring the peptide in 300 ml 50% ACN-water at pH 8 (adjusted by liquid ammonia) for 30 min at RT. The cyclized product was isolated by lyophilization. The Acm-protecting group was then removed by treating the peptide with a 5-fold molar excess of mercury(II)-acetate at pH 4-4.5 (adjusted with acetic acid) in 25% ACN-water. After stirring for 1 h at RT dithiotreitol (DTT) was added as a solid to the mixture in a 4-fold molar excess with respect to mercury(II)-acetate. The reaction was stirred for 2.5 h at RT and the precipitate that formed was removed by centrifugation. The supernatant was lyophilized to give the fully deprotected cyclized crude peptide.

The crude product was dissolved in 10% ACN-0.1% aq. TFA (50 ml) filtered and purified by preparative RP-HPLC. The column (Phenomenex Luna C18 10μ, 250×50 mm) was eluted at 50 ml/min with a gradient of 10 to 30% ACN in 0.1% aq TFA over 60 min. The desired peak fractions were pooled and lyophilized to afford 81 mg of pure peptide. Analytical RP-HPLC: t_{R}=17.8 min, purity 95% (Phenomenex Luna 5μ, 4.6×250 mm, 10-30% ACN in 0.1% aq TFA over 20 min at 1 ml/min, λ=215 nm). Electrospray MS: [M+H]⁺ of product expected at 1760.8 m/z, found at 1760.5 m/z.

### Example 2

cyclo-[CH₂CO-Lys-Arg-Gly-Val-Ile-Asp-Pro-Met-Arg-Cys]-Gly-Glu-Glu-Glu-Cys-NH₂

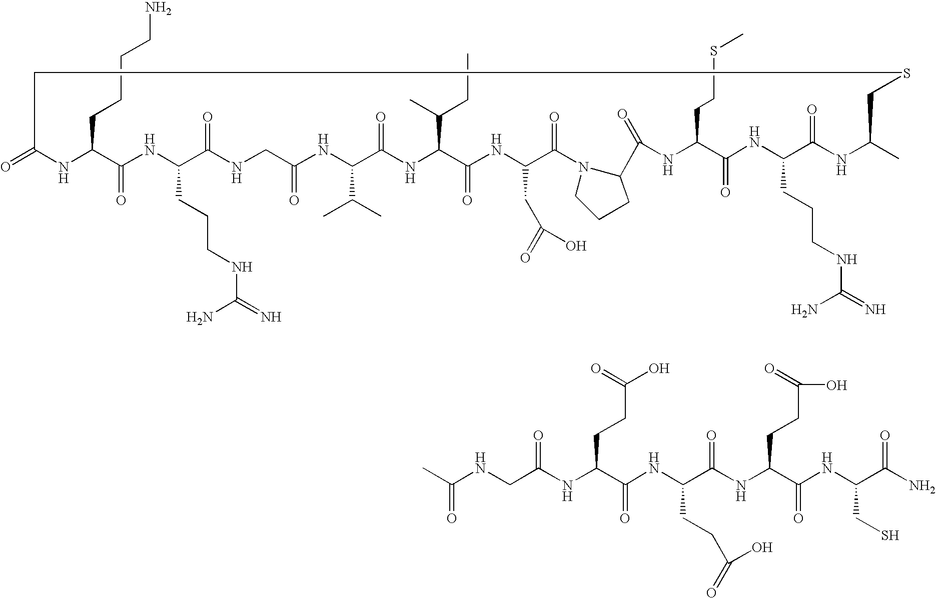

The peptidyl resin corresponding to the above sequence was assembled on a Rink Amide AM resin (0.74 mmol/g; from NovaBiochem) in a similar fashion to the corresponding peptidyl resin of Example 1. For additional side-chain protecting groups used see Example 1. The assembled peptidyl resin was bromoacetylated and worked up under the same conditions as described in Example 1.

Acidolytic deprotection of the peptide and subsequent cyclization was effected as described in Example 1 by treating an aliquot of the peptidyl resin (0.125 mmol) with TFA containing 2.5% ethanedithiol, 2.5% water and 1% triisopropylsilane for 2 hours. After precipitation from diethyl ether the thioetherbridge was formed in 500 ml 50% ACN-water at pH 8 over 40 min.

An aliquot of the crude cyclic Acm-protected peptide was purified by RP-HPLC as described in Example 1. Thus 165 mg were purified in 3 batches and the column (Vydac 218TP1022 10μ, 22×250 mm) was eluted at 10 m/min with a gradient of 10 to 30% ACN in 0.1% aq TFA over 40 min. The desired peak fractions were pooled and lyophilized to afford a yield of 12.5 mg. Final Acm-deprotection was carried out as outlined in Example 1 and the deprotected cyclic product was purified on a semi-prep RP-HPLC column (Phenomenex Luna, 10×250, 10μ using a gradient of 5-40% ACN in 0.1% aq TFA over 30 min affording 6 mg pure peptide. Analytical RP-HPLC: t_{R}=14.0 min, purity 99% (Phenomenex Luna 5μ, 4.6×250 mm, 5-50% ACN in 0.1% aq TFA over 20 min at 1 ml/min, λ=215 nm). Electrospray MS: [M+H]⁺ of product expected at 1760.8 m/z, found at 1760.5 m/z.

### Example 3

Ala-Glu-Gly-Glu-Phe-(cyclo-[Dpr-N^{β}(CH₂CO)-Arg-Val-Lys-Ile-Asp-Gly-Arg-Pro-Met-Cys])-Gly-Glu-Glu-Glu-Cys-NH₂

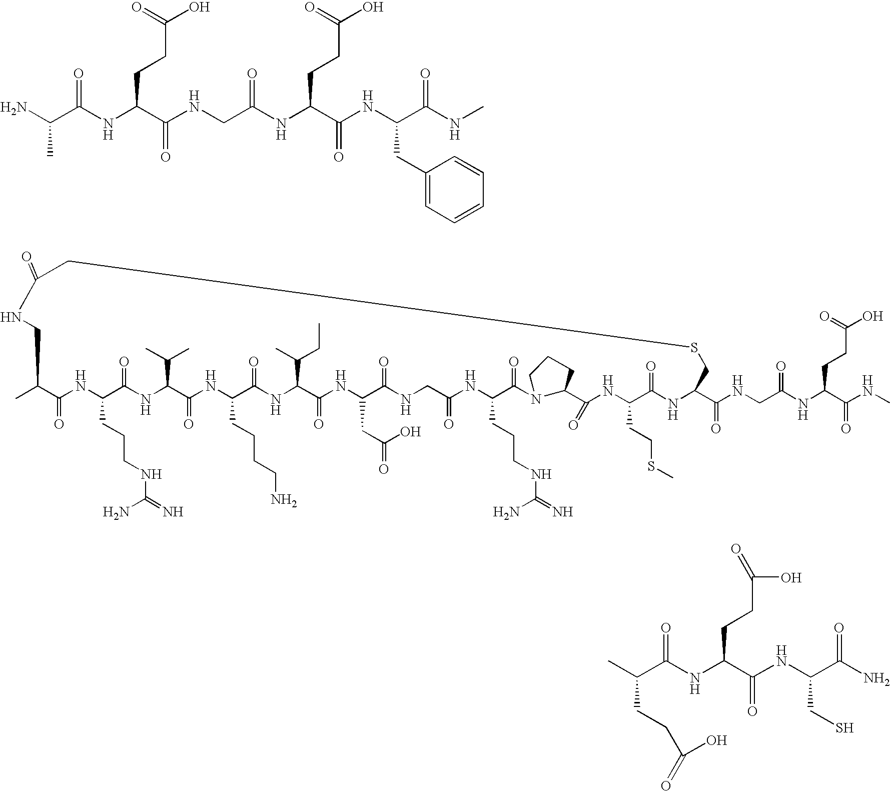

The peptidyl resin corresponding to the above sequence was assembled on a Rink Amide AM resin (0.74 mmol/g; from NovaBiochem) in a similar fashion to the corresponding peptidyl resin of Example 1. Additional side-chain protecting group used here was tert-butyl for Glu residues. The residues Dapa⁶ and Phe⁵ were introduced manually using the nitrogen bubbler apparatus as described in the folowing. A 5-fold molar excess of Fmoc-Dpr(ivDde)-OH was preactivated for 2 min with [7-Azabenzotriazol-1-yloxytris-(pyrrolidino)phosphonium-hexafluorophosphate] (PYAOP) and NMM in DMF and then added to the H-Ser-Tyr-Tyr-Ser-Asp-Gly-Val-Tyr-Asp-Cys]-Gly-Cys-Rink Amide resin (0.075 mmol). After 3 h a Kaiser test (Kaiser, E.; Colescott R. L.; Bossinger, C. D.; Cook, P. J., Anal. Biochemistry (1970) 84, p. 595) was negative, indicating complete reaction, and the resin was Fmoc-deprotected with 50% morpholine in DMF (2×15 min). The peptidyl resin was then transferred to a three-necked flask and treated with a 1.6-fold molar excess Fmoc-Phe-OH in DMF (2 ml) and a 3-fold excess NMM at −40° C. PyAOP (1.6-fold excess) in DMF (1.5 ml) was then added and the reaction mixture was let warm to RT. After stirring for 3.5 hrs the reaction was finished, and the final peptide assembly was completed on the automatic peptide synthesizer as described in Example 1. The peptidyl resin was then transferred to the nitrogen bubbler apparatus and the Dpr-N^{β} was deprotected by treatment with 2% hydrazine in DMF for 3×3 min. The liberated amino-function was then bromoacetylated as desribed in Example 1.

Acidolytic deprotection of the peptide was carried out as described in Example 1 by treating the resin with TFA containing 2.5% ethanedithiol, 2.5% water and 1% triisopropylsilane for 2 hrs. An aliquot of the crude product (50 mg) was cyclized by thioetherbridge formation in 100 ml 25% ACN-water at pH 8 and the final Acm-deprotection was carried out as described in Example 1.

The crude product was dissolved in 20% ACN-0.1% aq TFA (5 ml) filtered and purified by preparative RP-HPLC. The column (Phenomenex Luna C18 10μ, 22×250 mm) was eluted at 10 ml/min with a gradient of 10 to 25% ACN in 0.1% aq TFA over 40 min. The desired peak fractions were pooled and lyophilized to afford 5.3 mg of pure peptide. Analytical RP-HPLC: t_{R}=14.2 min, purity 90% (Phenomenex Luna 5μ, 4.6×250 mm, 10-40% ACN in 0.1% aq TFA over 20 min at 1 ml/min, λ=215 nm). Electrospray MS: [M+]²⁺ of product expected at 1190.5 m/z, found at 1190.4 m/z.

What is claimed is:

1. A targetable therapeutically active and/or diagnostic agent of formula (III)
V-L-Z (III)
wherein the vector V is a peptide comprising the amino acid sequence of formula (I)
Z¹-Arg-X²-X³-Ile-X⁵-X⁶-X⁷-X⁸-X⁹-Z²-Y¹ (I)
or formula (II)
Z¹-Arg-Val(Arg/Lys)Ile-Asp-Gly-X⁷-Pro-X⁹-Z²-Y¹ (II)
wherein
X² is an amino acid selected from the group Val, Leu, Ile and Tyr
X³ is an amino acid selected from the group Arg, Lys, Ile and Asn
X⁵ is an amino acid selected from the group A sp and Asn
X⁶ is an amino acid selected from the group Gly, Asn and Gln
X⁷ is an amino acid selected from the group Ala, Met, Gln, Arg, Glu and Val,
X⁸ is an amino acid selected from the group Pro, Gly, Ser and Arg
X⁹ is an amino acid selected from the group Ala, Met, Gln, Arg, Gly and Val
Z¹ represent an amino acid residue capable of forming a disulphide bond, preferably a cysteine or a homocysteine residue, or a residue capable of forming a thioether preferably the residue is Q-C(═O) wherein Q represents —(CH₂)n or —(CH₂)ₙ—C₆H₄ where n represents a positive integer 1 to 10 or is absent and
Z² represent an amino acid residue capable of forming a disulphide bond, preferably a cysteine or a homocysteine residue or is absent
Y¹ represents 1-10 amino acids or is absent
L represents a bond, a spacer or a linker and
Z represents an antineoplastic agent, a reporter or a group that optionally can carry an imaging moiety M.

2. A targetable therapeutically active and/or diagnostic agent according to claim 1 wherein the vector V is a peptide comprising the amino acid sequence

| | |
|---|---|
| Cys-Arg-Val-Arg-Ile-Asp-Gly-Ala-Pro- | (SEQ ID NO 1) |
| Ala-Cys,, | |
| Cys-Arg-Val-Arg-Ile-Asp-Asn-Met-Pro- | (SEQ ID NO 2) |
| Met-Cys,, | |
| Cys-Arg-Val-Arg-Ile-Asn-Gly-Gln-Pro- | (SEQ ID NO 3) |
| Gln-Cys,, | |
| Cys-Arg-Val-Lys-Ile-Asp-Gly-Arg-Pro- | (SEQ ID NO 4) |
| Met-Cys,, | |
| Cys-Arg-Leu-Lys-Ile-Asp-Gly-Met-Pro- | (SEQ ID NO 5) |
| Arg-Cys,, | |
| Cys-Arg-Ile-Lys-Ile-Asp-Gly-Glu-Gly- | (SEQ ID NO 6) |
| Gln-Cys,, | |
| Cys-Arg-Val-Tyr-Ile-Asp-Gly-Val-Ser- | (SEQ ID NO 7) |
| Val-Cys,, | |
| Cys-Arg-Val-Ile-Ile-Asp-Gly-Arg-Arg- | (SEQ ID NO 8) |
| Met-Cys,, | |
| Cys-Arg-Tyr-Asn-Ile-Asp-Gly-Arg-Pro- | (SEQ ID NO 9) |
| Gln-Cys, | |
| or | |
| Cys-Arg-Ile-Arg-Ile-Asp-Gln-Arg-Pro- | (SEQ ID NO 10) |
| Ala-Cys,. | |

3. An agent according to claim 1 where Z is a chelating agent of formula IV 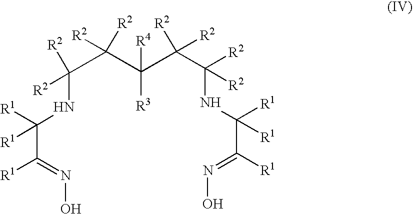
where:
each R¹, R², R³ and R⁴ is independently an R group;
each R group is independently H or C₁₋₁₀ alkyl, C₃₋₁₀ alkylaryl, C₂₋₁₀ alkoxyalkyl, C₁₋₁₀ hydroxyalkyl, C₁₋₁₀ alkylamine, C₁₋₁₀ fluoroalkyl, or 2 or more R groups, together with the atoms to which they are attached form a carbocyclic, heterocyclic, saturated or unsaturated ring.

4. An agent as claimed in claim 1 wherein Z comprises a reporter moiety M wherein the reporter moiety comprises metal radionuclides, paramagnetic metal ions, fluorescent metal ions, heavy metal ions or cluster ions.

5. An agent as claimed in claim 4 wherein the reporter moiety M comprises ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰³Pb, ¹⁴¹Ce or ¹⁸F.

6. An agent as claimed in claim 1 where each reporter (Z) can carry a multiplicity of vectors V.

7. An agent as claimed in claim 1 where the antineoplastic agent, Z represent cyclophosphamide, chloroambucil, busulphan, methotrexate, cytarabine, fluorouracil, vinblastine, paclitaxel, doxorubicin, daunorubicin, etoposide, teniposide, cisplatin, amsacrine or docetaxel.

8. A peptide comprising the amino acid sequence of formula (II)
Z¹-Arg-Val(Arg/Lys)Ile-Asp-Gly-X⁷-Pro-X⁹-Z²-Y^{1 (II)}
wherein
X⁷ is an amino acid selected from the group Ala, Met, Gln, Arg, Glu and Val,
X⁹ is an amino acid selected from the group Ala, Met, Gln, Arg, Gly and Val
Z¹ represent an amino acid residue capable of forming a disulphide bond, preferably a cysteine or a homocysteine residue, or a residue capable of forming a thioether preferably the residue is Q-C(═O) wherein Q represents —(CH₂)n or —(CH₂)ₙ—C₆H₄ where n represents a positive integer 1 to 10 or is absent and
Z² represent an amino acid residue capable of forming a disulphide bond, preferably a cysteine or a homocysteine residue or is absent
Y¹ represents 1-10 amino acids or is absent
or pharmaceutically acceptable salts thereof.

9. A peptide comprising the amino acid sequence

| | |
|---|---|
| Cys-Arg-Val-Arg-Ile-Asp-Gly-Ala-Pro- | (SEQ ID NO 1) |
| Ala-Cys,, | |
| Cys-Arg-Val-Arg-Ile-Asp-Asn-Met-Pro- | (SEQ ID NO 2) |
| Met-Cys,, | |
| Cys-Arg-Val-Arg-Ile-Asn-Gly-Gln-Pro- | (SEQ ID NO 3) |
| Gln-Cys,, | |
| Cys-Arg-Val-Lys-Ile-Asp-Gly-Arg-Pro- | (SEQ ID NO 4) |
| Met-Cys,, | |
| Cys-Arg-Leu-Lys-Ile-Asp-Gly-Met-Pro- | (SEQ ID NO 5) |
| Arg-Cys,, | |
| Cys-Arg-Ile-Lys-Ile-Asp-Gly-Glu-Gly- | (SEQ ID NO 6) |
| Gln-Cys,, | |
| Cys-Arg-Val-Tyr-Ile-Asp-Gly-Val-Ser- | (SEQ ID NO 7) |
| Val-Cys,, | |
| Cys-Arg-Val-Ile-Ile-Asp-Gly-Arg-Arg- | (SEQ ID NO 8) |
| Met-Cys,, | |
| Cys-Arg-Tyr-Asn-Ile-Asp-Gly-Arg-Pro- | (SEQ ID NO 9) |
| Gln-Cys, | |
| or | |
| Cys-Arg-Ile-Arg-Ile-Asp-Gln-Arg-Pro- | (SEQ ID NO 10) |
| Ala-Cys,. | |

10. A pharmaceutical composition comprising an effective amount of a compound of general Formula (III) or a salt thereof, together with one or more pharmaceutically acceptable adjuvants, excipients or diluents.

11. A method of generating enhanced images of a human or animal body previously administered with a contrast agent composition comprising a compound as claimed in claim 1, which method comprises generating an image of at least part of said body.
